(19) United States
(12) Patent Application Publication
(10) Pub. No.: US 2018/0037941 A1
(43) Pub. Date: Feb. 8, 2018

(54) METHOD FOR DETECTING GENETIC MUTATION

(71) Applicant: RIKEN GENESIS CO., LTD., Tokyo (JP)

(72) Inventors: Akio YAMANE, Tokyo (JP); Ryoko IMAGAWA, Tokyo (JP); Yuan YUAN, Tokyo (JP)

(73) Assignee: RIKEN GENESIS CO., LTD., Tokyo (JP)

(21) Appl. No.: 15/730,013

(22) Filed: Oct. 11, 2017

Related U.S. Application Data
(63) Continuation-in-part of application No. PCT/JP2016/062013, filed on Apr. 14, 2016.

(30) Foreign Application Priority Data
Apr. 14, 2015 (JP) 2015-082495

(51) Int. Cl. C12Q 1/68; C12N 15/10

(52) U.S. Cl. CPC C12Q 1/6827; C12N 15/1096; C12N 15/1031; C12Q 2600/156; C12Q 2533/101; C12Q 2535/10

(57) ABSTRACT

A method for detecting a genetic mutation is provided. The method comprising: carrying out PCR using a template DNA, a forward primer, a reverse primer and a wild-type oligonucleotide; and detecting an amplified DNA. In the method, the wild-type oligonucleotide comprises LNA or BNA, and on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

### TECHNICAL FIELD

The present invention relates to a method for detecting a genetic mutation using a PCR (polymerase chain reaction)-clamping method, the method being capable of detecting a mutant-type nucleic acid with a higher sensitivity by suppressing PCR amplification of a wild-type nucleic acid.

This application claims priority on the basis of Japanese Patent Application No. 2015-082495 filed on Apr. 14, 2015 in Japan, the disclosure of which is incorporated herein by reference.

### BACKGROUND ART

Due to the advancement of molecular target drugs in cancer fields, cancer treatment methods have been improved, and are approaching a major turning point. In particular, it has become possible to study the effect of a drug at a molecular level, and it has also become possible to predict the effect of a drug by examining a gene. For example, an anti-human epidermal growth factor receptor (hEGFR) antibody that is a therapeutic agent against colon cancer is not effective in patients having a mutation in the k-ras gene, and is effective only for patients having a wild-type k-ras gene. Therefore, in an actual clinical setting, genetic mutations of k-ras gene are examined before treatment. In addition, use of a molecular targeted drug against lung cancer is effective only for patients with having a mutation in the EGFR gene, and genetic mutations are examined in molecular targeted drug treatment against lung cancer as well. On the other hand, it has been evident that DNA released from cancer cells and cancer cells themselves are suspended in the blood of a cancer patient, and an attempt has been made to diagnose a cancer and predict the effect of a drug using the DNA and cancer cells suspended in the blood.

In examination of a genetic mutation as described above, it is necessary to detect a mutant gene with a relatively low abundance ratio, which is derived from cancer cells coexisting in normal cells. Particularly in treatment of lung cancer, cancer cells with a mutant gene resistant to a drug coexist at such an extremely low ratio that the mutant gene cannot be detected by a conventional examination method. Thus, there is the problem that in a cancer patient whose mutant gene has not been detected in examination before drug administration, the number of cancer cells having a resistant mutant gene in drug treatment gradually increases. If cancer cells having a resistant genetic mutation can be detected at a smaller abundance ratio as compared to conventional methods, the appearance of drug resistance can be predicted early so that better treatment can be performed.

From the above point of view, high-sensitivity detection of a cancer-related genetic mutation (detection of a mutant gene in a very low ratio, which is mixed in a wild-type gene) is increasingly important in cancer treatment.

The most typical method for examination of a genetic mutation is Sanger sequencing. Sanger sequencing has been considered to be a gold standard for detection of a genetic mutation. However, when the ratio of a mutant-type gene mixed in a wild-type gene is lower than 10 to 20%, it is difficult to detect the mutant gene. Thus, in cancer fields, a method with a sensitivity exceeding that of Sanger sequencing is required, various methods have been devised, and some of them have been used clinically (see Non-Patent Literature 1).

These methods include a method called PCR-clamping method. The method was first carried out using PNA (peptide nucleic acid) (see Non-Patent Literature 2). PNA is an artificial nucleic acid which is also called a peptide nucleic acid, and has a structure in which a phosphodiester bond connecting sugar moieties of the nucleic acid is replaced by a peptide bond having glycine as a unit. Unlike natural nucleic acids, PNA has no charge, but it is more strongly combined (hybridized) with a nucleic acid having a complementary sequence than DNA and RNA, and shows high specificity. Here, the high specificity means that PNA has high binding strength to fully complementary DNA or RNA, but when the nucleic acid includes even one base that is not complementary, the binding strength is considerably reduced. The PCR-clamping method is a method using a phenomenon in which when a PNA oligonucleotide (oligonucleotide containing PNA) having a sequence complementary to a wild-type gene is added in a PCR reaction, the PNA oligonucleotide is hybridized more specifically with a wild-type gene than to a mutant-type gene. In other words, the PCR-clamping method is a method for obtaining a PCR product in which amplification of a mutant-type gene with which a PNA oligonucleotide is not hybridized preferentially proceeds, so that the ratio of a mutant-type gene to a wild-type gene increases. In addition, non-natural nucleic acids which are more strongly hybridized with RNA and DNA than natural DNA, and used in the PCR-clamping method like PNA include LNA (locked nucleic acid) (See Non-Patent Literature 3). LNA is a compound in which 2′-oxygen and 4′-carbon in the ribose ring of a ribonucleoside are combined with each other by a methylene group to introduce a new cyclic structure, so that a change in steric structure of the ribose ring is restricted.

Non-natural nucleic acids that are strongly hybridized with RNA and DNA with high specificity as PNA and LNA include BNA (bridged nucleic acid) (see Patent Literature 1). Like LNA. BNA has a structure in which 2′-oxygen and 4′-carbon in the ribose ring of a ribonucleoside are cross-linked, but unlike LNA, BNA contains nitrogen atom in the linked structure, and has a six-membered ring as a ring structure. Further, it is easy to introduce a functional group via nitrogen atoms. It has also been reported that a k-ras mutation and an EGFR mutation were detected by the PCR-clamping method using a BNA oligonucleotide (oligonucleotide containing BNA) (see, for example, Patent Literature 2).

In addition, it has been reported that in a PCR-clamping method using a PNA oligonucleotide (oligonucleotide containing PNA), the positional relationship between the PNA oligonucleotide and a primer used for PCR influences the efficiency of PCR-clamping, i.e. the effect of suppressing wild-type nucleic acid amplification by the PNA oligonucleotide (see Non-Patent Literature 4).

The report shows that when on one of single-stranded DNA in two single-stranded DNAs to be a template DNA with which a PNA oligonucleotide is not hybridized, a region with which a primer is hybridized is more distant from a region complementary to a region with which the PNA oligonucleotide is hybridized, the efficiency of PCR-clamping is high.

### CITATION LIST

The Patent Literatures and Non-Patent Literatures are incorporated herein by reference.

### Patent Literatures

Patent Literature 1: JP 4731324 B2
Patent Literature 2: WO 2014/051076

### Non-Patent Literatures

Non-Patent Literature 1: Goto. et al., Annals of Oncology, 2012, vol. 23, pp. 2914-2919.
Non-Patent Literature 2: Thiede, et al., Nucleic Acids Research, 1996, vol. 24, p. 983-984.
Non-Patent Literature 3: Domingeuz, et al., Oncogene, 2005, vol. 24, p. 6830-4
Non-Patent Literature 4 Luo, et al., Nucleic Acids Research, 2006, vol. 34, e12.

### SUMMARY OF INVENTION

A first aspect of the present invention provides a method for detecting a genetic mutation, the method comprising: carrying out PCR using a template DNA comprising a target genetic mutation site, a forward primer and a reverse primer each configured to amplify a region containing the target genetic mutation site, and a wild-type oligonucleotide comprising a base sequence complementary to wild type template DNA; and detecting an amplified DNA comprising a mutant-type genetic mutation site on the basis of the result of PCR amplification, wherein the wild-type oligonucleotide comprises LNA or BNA, and wherein on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

A second aspect of the present invention provides method for detecting a genetic mutation, the method comprising: providing a sample comprising mutant type template DNA and wild type template DNA, wherein the mutant type template DNA comprises a mutant-type genetic mutation site, and the wild type template DNA comprises a wild-type genetic mutation site, selectively amplifying the mutant type template DNA in the sample using a forward primer and a reverse primer each configured to amplify a region containing the mutant type genetic mutation site, and a wild-type oligonucleotide comprising a base sequence complementary to wild type template DNA; and detecting an amplified DNA comprising a mutant-type genetic mutation site on the basis of the result of PCR amplification, wherein the wild-type oligonucleotide comprises LNA or BNA, and wherein on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

A third aspect of the present invention provides a method for amplifying DNA, the method comprising: providing a sample comprising mutant template DNA and wild type template DNA, wherein the mutant template DNA comprises a mutant-type sequence comprising a mutation, and the wild type template DNA comprises a wild-type sequence, and selectively amplifying the mutant template DNA in the sample using, a forward primer and a reverse primer each configured to amplify a region containing the mutation, and a wild-type oligonucleotide complementary to wild type sequence; wherein the wild-type oligonucleotide comprises LNA or BNA, and wherein on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

### DESCRIPTION OF EMBODIMENTS

A method for detecting a genetic mutation according to a first embodiment of the present invention is a PCR-clamping method in which an oligonucleotide containing a non-natural nucleic acid composed of at least one LNA or BNA is used as a wild-type oligonucleotide, and the positional relationship between the wild-type oligonucleotide and a primer used for PCR is set within a specific range to improve the efficiency of PCR-clamping. In the PCR-clamping method, by carrying out PCR in the presence of a wild-type oligonucleotide having a base sequence complementary to a genome region in which the targeted site is wild-type, PCR amplification of a wild-type nucleic acid (nucleic acid in which genetic mutation site is a wild-type genetic mutation site) is suppressed to increase the detection sensitivity of a mutant-type nucleic acid (a nucleic acid in which the genetic mutation site is a mutant-type site). The positional relationship between the wild-type oligonucleotide and the primer influences the efficiency of PCR-clamping. The positional relationship suitable for improving the efficiency of PCR-clamping varies depending on the kind of non-natural nucleic acid possessed by the wild-type oligonucleotide. In the method for detecting a genetic mutation according to this embodiment, an oligonucleotide containing LNA or BNA is used as a wild-type oligonucleotide, and the distance between the wild-type oligonucleotide and the primer on a template DNA is set to 18 bases or less. The distance between the oligonucleotide containing LNA or BNA and the primer is made as small as 18 bases or less, whereby the efficiency of PCR-clamping can be remarkably improved. It has been known that the distance between the wild-type oligonucleotide and the primer influences the efficiency of PCR-clamping. However, it is the finding discovered for the first time by the present inventors that a distance suitable for improving the efficiency of PCR-clamping really varies depending on the kind of non-natural nucleic acid possessed by the wild-type oligonucleotide.

The wild-type oligonucleotide to be used in the method for detecting a genetic mutation according to this embodiment has a base sequence complementary to a genome region in which the target genetic mutation site to be detected is a wild-type. That is, the wild-type oligonucleotide contains bases that are not fully complementary to the mutant-type nucleic acid in which the target genetic mutation site is a mutant-type. Thus, the wild-type oligonucleotide is more specifically hybridized with a wild-type nucleic acid than with a mutant-type nucleic acid.

The wild-type oligonucleotide also contains at least one LNA or BNA. LNA and BNA have a structure in which nucleosides are linked by a phosphodiester bond, and this structure can be said to be much closer to a natural nucleic acid as compared to PNA. The PNA oligonucleotide has some problems that it is difficult to synthesize a PNA oligonucleotide with a large chain length because of the character of its structure, or the solubility in water is significantly reduced depending on the base sequence. On the other hand, the LNA oligonucleotide and the BNA oligonucleotide do not have such a problem, and can be said to be substances more suitable for the PCR-clamping method. The nucleoside structure of BNA is shown in the following formulae (1) and (2).

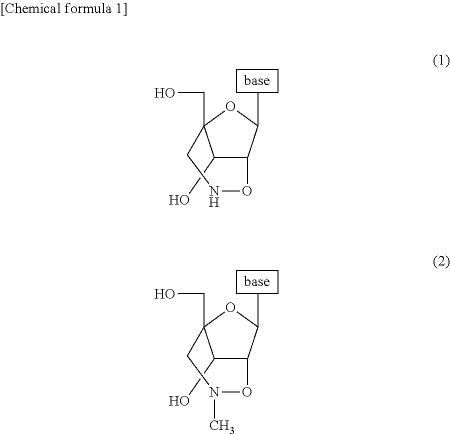

The wild-type oligonucleotide is a LNA oligonucleotide or a BNA oligonucleotide. Therefore, the wild-type oligonucleotide has higher binding strength to a template DNA and higher specificity as compared to an oligonucleotide formed only from a natural nucleic acid such as DNA or RNA, and a PNA oligonucleotide. Therefore, the wild-type oligonucleotide is strongly combined (hybridized) with a wild-type nucleic acid, but hardly hybridized with a mutant-type nucleic acid. Therefore, PCR amplification with a wild-type nucleic acid as a template can be specifically suppressed. That is, by PCR-amplifying a region containing a target genetic mutation site in the presence of a wild-type oligonucleotide, the ratio of the mutant-type nucleic acid in the PCR amplification product can be made higher than the ratio of the mutant-type nucleic acid in the template DNA. Such an effect of suppressing amplification of a wild-type nucleic acid to increase the ratio of the mutant-type nucleic acid in the PCR amplification product may be referred to as a “mutation enrichment effect” or a “wild-type amplification suppression effect”.

The wild-type oligonucleotide to be used in the method for detecting a genetic mutation according to this embodiment may be a LNA oligonucleotide, a BNA oligonucleotide, or an oligonucleotide including both LNA and BNA. The wild-type oligonucleotide is preferably a BNA oligonucleotide because it has higher bonding strength and specificity to a template DNA, and attains a higher mutation enrichent effect.

The wild-type oligonucleotide may include only at least one of LNA and BNA. The wild-type oligonucleotide may include at least one of LNA and BNA and a natural nucleic acid (at least one of DNA and RNA). In addition, when the wild-type oligonucleotide includes at least one of LNA and BNA and a natural nucleic acid, the wild-type oligonucleotide may contain two or more LNAs, or contain two or more BNAs, or contain both LNA and BNA. Protecting groups for synthesizing a nucleic acid by a phosphoramidite method and activated phosphate groups (amidite) can be introduced in the BNA nucleoside as with a natural nucleoside, and a BNA oligonucleotide can be synthesized as with usual oligonucleotide synthesis. In addition, there is only one kind of LNA having a structure in which 2′-oxygen and 4′-carbon in the ribose ring of a ribonucleoside are combined with each other by a methylene group. A nucleoside unit obtained by combining the structure with four kinds of bases can be mixed with a natural nucleoside as with BNA to synthesize a LNA oligonucleotide.

A site which is hybridized with a target genetic mutation site in a wild-type oligonucleotide, i.e. a site which is non-complementary to a mutant-type nucleic acid can be appropriately determined with consideration given to, for example, the kind of genetic mutation and the base sequence of a genome region containing a target genetic mutation site so that nonspecific hybridization with the mutant-type nucleic acid is sufficiently suppressed. For example, the base sequence of a wild-type oligonucleotide is designed using known primer design software or the like in such a manner that provided that all the wild-type oligonucleotide is an oligonucleotide formed only from DNA, the Tm value of the oligonucleotide and the wild-type nucleic acid is sufficiently higher than the Tm value of the oligonucleotide and the mutant-type nucleic acid. A wild-type oligonucleotide can be designed by replacing one or more bases in the designed base sequence by BNA or LNA.

Since the wild-type oligonucleotide is hybridized with a template DNA, an extending reaction may be occurred by a DNA polymerase. However, even if the extending reaction is occurred, the extented product having BNA or LNA reduces the activity of the polymerase as a template for DNA polymerase. Therefore, efficiency of the extended product to be template is low, and thus there is no particular problem.

In particular, when BNA or LNA is contiguous, it is hard to be a template for DNA polymerase.

The wild-type oligonucleotide may be protected at the 3′ terminal hydroxyl group with a substituent so as not to cause an extending reaction. Examples of the substituent include a phosphoric acid group. However, some DNA polymerases to be used in PCR have a 3′→5′ nuclease activity that performs a repair function. Therefore, depending on the substituent, the oligonucleotide may be decomposed to promote an extending reaction. Accordingly, it is preferable that the substituent to be introduced into the wild-type oligonucleotide at the 3′ terminal hydroxyl group determined in consideration of a DNA polymerase to be used.

It is preferable that the site that is hybridized with the target genetic mutation site in the wild-type oligonucleotide used in this embodiment is not the 5′ terminal or the 3′ terminal in the wild-type oligonucleotide, and it is more preferable that this site is in the vicinity of center of the wild-type oligonucleotide. In particular, when the genetic mutation is a single-base mutation, it is preferable that there is a site that is hybridized with the genetic mutation site in the vicinity of the center of the wild-type oligonucleotide.

The genetic mutation to be detected in this embodiment is not particularly limited, and may be a mutation in which one or more bases may be substituted, deleted or inserted with respect to the wild-type base sequence.

In the method for detecting a genetic mutation according to this embodiment, a double-stranded DNA containing a base sequence identical to that of a genome region containing a target genetic mutation site is used as a template DNA, and a forward primer and a reverse primer for amplifying a region containing the genetic mutation site by PCR are used to carry out PCR in the presence of the wild-type oligonucleotide. Hereinafter, among two single-stranded DNAs forming a template DNA, a single-stranded DNA with which a wild-type oligonucleotide is hybridized when the template DNA is a wild-type DNA is referred to as a “first single-stranded template DNA”, and the other single-stranded DNA is referred to as a “second single-stranded template DNA”. In addition, of the forward primer and the reverse primer, a primer which has a base sequence complementary to a part of the first single-stranded template DNA and which is hybridized with the first single-stranded template DNA is referred to as a “first primer, and a primer which has a base sequence complementary to a part of the second single-stranded template DNA and which is hybridized with the second single-stranded template DNA is referred to as a “second primer”. In the template DNA, the region with which a wild-type oligonucleotide is hybridized lies in a region sandwiched between a region that is hybridized with the first primer and a region that is hybridized with the second primer.

In the method for detecting a genetic mutation according to this embodiment, the region with which the wild-type oligonucleotide is hybridized and the region with which the first primer is hybridized, on the first single-stranded template DNA, partially overlap each other. Alternatively, these regions are separated by 1 to 18 bases. The wild-type oligonucleotide is an oligonucleotide containing at least one of BNA and LNA. Therefore, by reducing the distance between the region with which the wild-type oligonucleotide is hybridized with and the region with which the first primer is hybridized on the first single-stranded template DNA, the efficiency of PCR-clamping by the wild-type oligonucleotide (i.e. mutation enrichment effect) can be further improved. In this embodiment, the distance between the 5′ terminal of the wild-type oligonucleotide and the 3′ terminal of the first primer on the first single-stranded template DNA is preferably −5 bases to 18 bases, more preferably −2 bases to 10 bases, still more preferably 1 to 5 bases. The distance of “−X bases” means that the 5′ terminal of the wild-type oligonucleotide and the 3′ terminal of the first primer overlap each other by X bases.

Each of the first primer and the second primer to be used in this embodiment may be an oligonucleotide having a nucleoside forming a natural DNA, the oligonucleotide containing one or more kinds of non-natural bases in a part thereof. Examples of the non-natural base include LNA and BNA. By using a primer containing a non-natural base, binding to the template DNA can be strengthened, and base specificity can be improved.

However, since a non-natural base may hinder the extending reaction by a DNA polymerase, it is preferable that the non-natural base exists on the 3′ terminal of the primer. In addition, for detection of the amplification product obtained by PCR amplification, a marker substance may be combined to the primer and used as a labeling. The marker substance may be a substance, which is specifically bind with a specific protein like biotin etc., or a substance which can be directly detected like a fluorescent substance etc.

The first primer and the second primer can be designed using known software on the basis of the base sequence of a genome region containing a target genetic mutation site so that nucleic acid fragments containing a target genetic mutation site can be amplified. Specifically, these primers are designed with consideration given to the Tm value, which is an index of binding strength between the primer and the template DNA, formation of a secondary structure in the primer, binding between primers, binding strength of the primer to the nonspecific region of the genome, and the like. The base length of the first primer and the second primer can be set to, for example, 15 to 30 bases as with a primer that is generally used for PCR.

The length of the nucleic acid fragment to be PCR-amplified by the first primer and the second primer is not particularly limited, and may be determined in consideration of how the amplified nucleic acid fragment is used. For example, the length of the nucleic acid fragment may be a length equivalent to 40 to several thousand bases, and is more preferably a length equivalent to 40 to 500 bases.

In PCR, a mixture of a first primer and a second primer, a wild-type oligonucleotide, a template DNA, four kinds of deoxynucleotide triphosphates and a heat-resistant DNA polymerase with a buffer solution is used as a reaction solution. Further, PCR is carried out by increasing or decreasing the liquid temperature of the reaction solution in accordance with the temperature cycle. The deoxynucleotide triphosphates, heat-resistant DNA polymerase and buffer solution to be used can be appropriately selected from structures that are generally used in PCR. The concentration of the first primer and the second primer in the reaction solution can be set to a concentration comparable to the concentration of a primer that is used in general PCR. In addition, the amount of the wild-type oligonucleotide in the reaction solution is not particularly limited as long as the effect of PCR-clamping is obtained, and for example, the amount of the wild-type oligonucleotide may be ⅓ of the amount of the first primer or the second primer. When the amount of the template DNA is excessively small or the effect of PCR-clamping by the wild-type oligonucleotide is excessively high, an amplification product having a base sequence that has not been present in the original template may be obtained due to an error of the DNA polymerase. In this case, the problem can be avoided by, for example, reducing the number of PCR cycles, or using a heat-resistant DNA polymerase with a low error rate.

The double-stranded DNA containing a base sequence identical to a genome region containing a target genetic mutation site to be used as a template DNA may be the genome DNA itself, or an amplification product obtained by amplifying a genome region containing a target genetic mutation site beforehand by PCR using a genome DNA as a template. The genome DNA can be extracted from cells or tissues by a usual method.

The cells or tissues collected from a subject in order to acquire a genome DNA are not particularly limited as long as they contain a genome DNA, and they may include any cells or tissues. Since it is desirable that collection of cells or tissues is minimally invasive for the subject, for example, blood, blood cell components, a lymph fluid, semen, hair and the like are preferable, and blood and blood cell components as fractions of blood (e.g. leukocytes) are more preferable. In addition, when the target genetic mutation site is situated in a coding region of a structural gene, a cDNA synthesized by reverse transcription using a total RNA as a template extracted from cells or tissues collected from the subject can be used as a template DNA.

In the PCR, the nucleic acid chain-extention reaction using a DNA polymerase is carried out at a temperature at which the wild-type oligonucleotide is hybridized with a template DNA in which the genetic mutation site is a wild-type, and the wild-type oligonucleotide is not hybridized with a template DNA in which the genetic mutation site is a mutant-type. That is, the temperature in the extention reaction is a temperature at which the first primer and the first single-stranded template DNA can be hybridized with each other, the wild-type oligonucleotide and the wild-type first single-stranded template DNA can be hybridized with each other, the second primer and the second single-stranded template DNA can be hybridized with each other, and the wild-type oligonucleotide and the mutant-type first single-stranded template DNA are not hybridized with each other. An extending reaction of heat-resistant DNA polymerase that is generally used in PCR is most efficiently carried out at about 68 to 72° C. Therefore, it is preferable that the wild-type oligonucleotide to be used in this embodiment is designed in such a manner that the Tm value with the wild-type first single-stranded template DNA is 68° C. or higher, and the Tm value with the mutant-type first single-stranded template DNA is lower than 68° C.

The Tm value is an index of the stability of double-strand formation, i.e. the binding strength of oligonucleotide with the template DNA. The Tm value of an oligonucleotide formed from a natural nucleoside can be predicted by calculation on the basis of a value obtained from an empirical value, and is generally determined by calculation. For the Tm value of a LNA oligonucleotide and a template DNA (single-stranded DNA formed from a natural nucleoside), a calculation formula similar to that for the natural nucleoside has been devised, and the Tm value for the LNA oligonucleotide can be predicted using such a calculation formula. The Tm value thus obtained is referred to in the design of a primer or the like. However, the calculated Tm value is merely a predicted value, and the actual Tm value is not necessarily consistent with the predicted value for an oligonucleotide formed from only a natural nucleoside, and a LNA oligonucleotide.

Currently, there is no reliable formula for predicting the Tm value of a BNA oligonucleotide and a template DNA. BNA has a structure different from that of LNA, and for BNA, there are two kinds of nucleosides of formula (1) or formula (2). Thus, it is extremely difficult to derive a calculation formula. Thus, there is no method for predicting the Tm value of a BNA oligonucleotide except for a method in which the Tm value is very roughly predicted, and an experiment is actually conducted to make a measurement.

In the method for detecting a genetic mutation according to this embodiment, a double-stranded DNA in which the genetic mutation site in the template DNA is a mutant-type is detected on the basis of the total amount of the resulting PCR amplification product or the amount of a mutant-type nucleic acid in the PCR amplification product. For example, when the total amount of the PCR amplification product in the number of cycles before the amplification product reaches the plateau is larger than the total amount of the PCR amplification product in the same number of cycles where PCR is carried out beforehand under the same conditions using a template DNA in which all of nucleic acids are wild-type nucleic acids, it can be determined that a mutant-type nucleic acid is present in the template DNA. In addition, when a mutant-type nucleic acid is present in the resulting PCR amplification product, it can be determined that a mutant-type nucleic acid is present in the template DNA.

Quantitative PCR can be used as PCR that is carried out in the method for detecting a genetic mutation according to this embodiment. Real-time PCR is preferable because it is easy to measure the amount of the PCR amplification product. In this embodiment, a case where real-time PCR is carried out will be described. The method for carrying out real-time PCR is a method in which the amount of DNA amplification is detected in real time using a fluorescent reagent, and the method requires a device in which a thermal cycler and a fluorescence spectrophotometer are integrated. Such a device is commercially available. There are several methods depending on the fluorescent reagent to be used, and typical examples thereof include an intercalator method and a hydrolysis probe method.

The intercalator method is a method making use of a phenomenon in which an intercalator added in a reaction solution for PCR binds with a generated double-stranded DNA and emits fluorescence, thereby fluorescence increases as the number of amplified DNA fragments increases. By measuring the fluorescence intensity with the reaction solution irradiated with excitation light, the generation amount of the amplification product can be monitored.

The hydrolysis probe method is a method using an oligonucleotide which is labeled with a fluorescent substance and a quenching substance, and contains a base sequence complementary to a PCR amplification product to be detected. The hydrolysis probe does not emit fluorescence by FRET (fluorescence resonance energy transfer) as it is. The hydrolysis probe is hybridized with the PCR amplification product during annealing, and decomposed by exonuclease activity of DNA polymerase during extention reaction, so that a fluorescent substance is released to emit fluorescence. Therefore, the generation amount of the amplification product can be monitored by measuring the fluorescence intensity. The fluorescent substance and quenching substance which label the hydrolysis probe can be appropriately selected from a combination of fluorescent substances and quenching substances usable for FRET. Examples of the fluorescent substance usable for FRET include FAM, Yakima Yellow (registered trademark), fluorescein, FITC and VIC (registered trademark). In addition, examples of the quenching substance usable for FRET include TAMRA.

The effect (magnitude of efficiency) of PCR-clamping of the wild-type oligonucleoLide can be determined from the following formula.

ΔΔCt=ΔCt(wild-type){Ct(wild-type: wild-type oligonucleotide present)−Ct (wild-type: no wild-type oligonucleotide)}−ΔCt(mutant-type){Ct(mutant-type: wild-type oligonucleotide present)−Ct(mutant-type: no wild-type oligonucleotide)}

Here, “Ct” is the number of cycles at which a fluorescence value (threshold) above a certain level is obtained when real-time PCR is carried out in fluorescence measurement. “ΔCt (wild-type)” is an index that indicates how much amplification of PCR is hindered by addition of a wild-type oligonucleotide when only a wild-type nucleic acid is used as a template DNA. The value of ΔCt (wild-type) is preferably as large as possible. On the other hand, “ΔCt (mutant-type)” is an index that indicates how much amplification of PCR is hindered by addition of a wild-type oligonucleotide when only a mutant-type nucleic acid is used as a template DNA. The value of ΔCt (mutant-type) is preferably as small as possible. For obtaining a high effect of PCR-clamping, it is preferable that ΔCt (wild-type) is large, and ΔCt (mutant-type) is small. That is, ΔΔCt is preferably large. By using ΔΔCt as an index, the effect of PCR-clamping using a wild-type oligonucleotide can be appropriately evaluated. ΔΔCt can be determined using either the intercalator method or the hydrolysis probe method. However, when the hydrolysis probe method is used, a hydrolysis probe that binds with both a wild-type nucleic acid and a mutant-type nucleic acid must be used.

For example, in the case of using the intercalator method, real-time PCR is carried out in the presence of a wild-type oligonucleotide, so that the Ct value with only a wild-type nucleic acid as a template DNA is determined in advance. When the Ct value of a template DNA as a subject for which presence/absence of a genetic mutation is examined is smaller than the above-mentioned Ct value, it can be determined that a mutant-type nucleic acid has been present in the template DNA. When the hydrolysis probe method is carried out using a hydrolysis probe that bind with both a wild-type nucleic acid and a mutant-type nucleic acid, real-time PCR is carried out in the presence of a wild-type oligonucleotide, so that the Ct value with only a wild-type nucleic acid as a template DNA is determined in advance as in the case of the intercalator method. When the Ct value of a template DNA as a subject for which presence/absence of a genetic mutation is examined is smaller than the above-mentioned Ct value, it can be determined that a mutant-type nucleic acid has been present in the template DNA.

When the hydrolysis probe method is carried out using a hydrolysis probe which binds specifically with a mutant-type nucleic acid, it can be determined that a mutant-type nucleic acid has been present in the template DNA in the case where an amplification product as a mutant-type nucleic acid is detected. In the method for detecting a genetic mutation according to this embodiment, a mutation enrichment effect is obtained with a wild-type oligonucleotide, and therefore a mutant-type nucleic acid in an amplification product can be detected with a higher sensitivity by a hydrolysis probe which binds specifically with a mutant-type nucleic acid.

A enriched mutant-type nucleic acid can be detected using any of various methods other than the intercalator method and the hydrolysis probe method. Therefore, in this embodiment, the amount of the mutant-type nucleic acid in the PCR amplification product can be measured by a method which is appropriately selected from known measurement methods with consideration given to convenience, sensitivity, cost, carry-over contamination derived from a PCR amplification product, and so on. Examples of the method for detecting a mutant-type nucleic acid in an amplification product include a Sanger sequencing method. When the Sanger sequencing method is used alone, the mutation detection sensitivity is not high. However, in the method for detecting a genetic mutation according to this embodiment, the ratio of a mutant-type nucleic acid in the amplification product is raised by the mutation concentration effect. Therefore, even when the ratio of the mutant-type nucleic acid in the original specimen is low, the mutant-type nucleic acid in the amplification product can be detected by the Sanger sequencing method. In addition, by using a melting curve analysis method, a concentrated mutant-type nucleic acid can be easily detected.

A kit for detecting a genetic mutation according to a second embodiment of the present invention includes the wild-type oligonucleotide, the first primer and the second primer. By using the kit for detecting a genetic mutation, the method for detecting a genetic mutation according to the first embodiment can be more conveniently carried out.

The kit for detecting a genetic mutation according to this embodiment may further include a buffer solution for preparing a PCR reaction solution, a heat-resistant DNA polymerase, four kinds of deoxynucleotide triphosphates, a kit for detecting the genetic mutation, and so on.

### EXAMPLES

Hereinafter, the present invention will be described in detail by way of examples, but the present invention is not limited to the following examples.

### <Primer and BNA Oligonucleotide>

Primers were synthesized by Operon Biotechnologies Inc. (now Eurofins Genomics K. K.) on request, and purified with a cartridge, and these primers were used in the following experiments.

In addition, BNA oligonucleotides were synthesized by GeneDesign Inc. on request, and purified by reverse phase HPLC, and these BNA oligonucleotides were used. Hereinafter, the BNA oligonucleotide may be abbreviated as a BNA oligo.

In the following examples, the base sequence of the BNA oligonucleotide used was described in accordance with the following notation system. Specifically, among those having the BNA structure represented by the above formula (2), one including an adenine base is described as A(E), one including a guanine base is described as G(E), one including a 5-methylcytosine base is described as 5(E), and one including a thymine base is described as T(E). In addition, among those having the BNA structure represented by the above formula (1), one including thymine is represented described as T(H), and one including 5-methylcytosine is described as 5(H). Further, “p” is added when a phosphate group is introduced to the 3′-hydroxyl group at the 3′ terminal of the BNA oligonucleotide. Natural nucleosides are described as A, G, C and T.

### <Plasmid>

An artificially synthesized plasmid was cut by a restriction enzyme Hind III, and linearized to obtain a template DNA for PCR, and the template DNA thus obtained was used in the following experiments. 20,000 to 30,000 copies were used per PCR reaction. The plasmid was one prepared by inserting fragment sequences of hEGFR into a restriction enzyme EcoRV site of pMD20T. The plasmid was purchased from Takara Bio Inc.

The name of the plasmid, the base sequence of the inserted hEGFR fragment and its SEQ ID NO, thereof are shown in Table 1. “EG-” in the name of the plasmid is followed by the genotype of the EGFR fragment inserted into the plasmid.

TABLE 1

| Name | SEQ ID NO. |
|---|---|
| EG_G719wt | 1 |
| EG_G719C | 2 |
| EG_G719S | 3 |
| EG_G719A | 4 |
| EG_19wt | 5 |
| EG_del746_750 (2236) | 6 |
| EG_del746_750 (2235) | 7 |
| EG_delL747_A750insP | 8 |
| EG_delL747_S752E746V | 9 |
| EG_delL747_A753insS | 10 |
| EG_del752_759 (2253) | 11 |
| EG_20wt | 12 |
| EG_T790M | 13 |
| EG_21wt | 14 |
| EG_L858R | 15 |
| EG_L861Q | 16 |

### <Real-Time PCR>

For real-time PCR, the SYBR Green method was employed, and a commercially available kit “Takara SYBR Premix Dimer Eracer (Takara Bio Inc.)” was used. The composition of the PCR reaction solution is shown in Table 2. When a BNA oligonucleotide was not added, H₂O in a volume corresponding to that of the BNA oligonucleotide was added to adjust the total amount of the reaction solution.

TABLE 2

| | Volume | |
|---|---|---|
| 10 μM Forward primer | 0.6 | μL |
| 10 μM Reverse primer | 0.6 | μL |
| 5 μM Oligonucleotide | 0.4 | μL |
| Takara SYBR Premix DimerEraser (RR091A) | 10 | μL |
| H₂O | 5.4 | μL |
| Plasmid solution | 3 | μL |
| Total | 20 | μL |

As the real-time PCR apparatus. “CFX 96 real-time PCR detection system (manufactured by BioRad Company)” was used. As reaction conditions, 50 cycles of reaction were carried out with one cycle including, a reaction at 95° C. (for 20 seconds), a reaction at 58° C. (for 30 seconds), and a reaction at 72° C. (for 30 seconds) after an initial reaction at 95° C. (for 30 seconds).

In addition, the Ct value was calculated by an automatic calculation method using an apparatus, and the ΔΔCt value was calculated in accordance with the formula described above.

### Example 1

Using the primers and BNA oligonucleotide shown in Table 3, the mutation concentration effect (ΔΔCt) by PCR-clamping in detection of a deletion mutation in exon 19 of hEGFR was examined. SEQ ID NO: 22 is the number of a base sequence before the BNA oligo (E19B4) is subjected to various kinds of modifications.

As template DNAs, EG_19 wt as a wild-type plasmid and EG_delL747_T751insS as a plasmid having a L747T751>S mutation were used. The reverse primer is hybridized with a single-stranded template DNA with which a BNA oligonucleotide is hybridized, and the forward primer is hybridized with a single-stranded template DNA complementary to the single-stranded template DNA with which the BNA oligonucleotide is hybridized.

TABLE 3

| | Base sequence | SEQ ID NO. |
|---|---|---|
| Forward primer | 5′-TCTCTGTCATAGGGACTCTGGA | 17 |
| (E19F1) | | |
| Forward primer | 5′-AGTTAAAATTCCCGTCGC | 18 |
| (delF7) | | |
| Reverse primer | 5′-GAGAAAAGGTGGGCCTGAGG | 19 |
| (E19R1) | | |
| Reverse primer | 5′-GATTTCCTTGTTGGCTTTCG | 20 |
| (delR3) | | |
| Reverse primer | 5′-AGAGCAAAGGAGAAACTC | 21 |
| (E19R2) | | |
| BNA oligo | 5'-GT(H)T(H)GCT(H)T(H)5(H)T(H)5 | (22) |
| (E19B4) | (H)TT(H)AA(E)TTCCp | |

Table 4 shows combinations of primers, and ΔΔCt values when the BNA oligo (E19B4) is used. In Table 4, the “wild-type ΔCt” means ΔCt when the wild-type plasmid EG-19 wt is used, and the “mutant-type ΔCt” means ΔCt when the mutant-type plasmid EG-delL747-T751insS is used.

TABLE 4

| | | ΔCt for | ΔCt for | |
|---|---|---|---|---|
| Forward primer | Reverse primer | wild-type | mutant-type | ΔΔCt |
| delF7 | delR3 | 14.4 | −0.1 | 14.5 |
| E19F1 | E19R1 | −0.2 | 0.4 | −0.6 |
| delF7 | E19R1 | 0.7 | 0 | 0.7 |
| E19F1 | delR3 | 14.6 | −0.2 | 14.8 |
| E19F1 | E19R2 | 1.6 | −0.1 | 1.7 |

From these results, it was revealed that the mutation concentration effect by the BNA oligo (E19B4) was dramatically influenced by a combination of primers. It was found that only when the reverse primer was delR3, a large ΔΔCt value of 10 or more was obtained, a mutation concentration effect was exhibited, and the mutation concentration effect was not influenced by the forward primer. On the template DNA, the 5′ terminal of the BNA oligo (E19B4) is separated from the 3′ terminal of the reverse primer delR3 by 5 bases. In addition, the 5′ terminal of the BNA oligo (E19B4) is separated from the 3′ terminal of the reverse primer E19R2 by 33 bases, and separated from the 3′ terminal of the reverse primer E19R1 by 72 bases. In addition, on the template DNA, the base complementary to the 3′ terminal of the BNA oligo (E19B4) is separated from the 3′ terminal of the forward primer delF7 by 6 bases, and separated from the 3′ terminal of the forward primer E19F1 by 42 bases. That is, when the distance between the 5′ terminal of the BNA oligo (E19B4) and the 3′ terminal of the primer that is hybridized with a single-stranded template DNA identical to the single-stranded template DNA with which the BNA oligo (E19B4) is hybridized was 72 bases or 33 bases, the wild-type amplification suppression effect (mutation concentration effect) by the BNA oligo (E19B4) was not obtained at all. On the other hand, when the distance was 5 bases, a remarkable effect was obtained. In addition, it was revealed that the number of bases between the BNA oligo (E19B4) and the primer that is hybridized with a single-stranded template DNA complementary to the single-stranded template DNA with which the BNA oligo (E19B4) is hybridized did not influence the mutation concentration effect by the BNA oligo (E19B4) at all. That is, these results showed that for the mutation concentration effect by the BNA oligonucleotide, the distance between the BNA oligonucleotide and a primer that is hybridized with the same single-stranded template DNA was extremely important.

### Example 21

Using a combination of a primer set (forward primer E19F1 and reverse primer delR3) and a BNA oligo (E19B4) with which a remarkable mutation concentration effect was exhibited in Example 1, whether the same effect was obtained for another deletion mutation present in the same genome region was examined. For each mutation, plasmid EG_de1746_750(2235), plasmid EG_de1746_750(2236), plasmid EG_DelL747_P753 insS, plasmid EG_delL747_A750insP, plasmid EG_L747_S752 E746V were used as template DNAs. The obtained ΔΔCt values are shown in Table 5.

TABLE 5

| Plasmid as template | ΔΔCt |
|---|---|
| del746_750 (2235) | 15.3 |
| del746_750 (2236) | 15.2 |
| DelL747_P753insS | 14.2 |
| delL747_A750insP | 15.1 |
| L747_S752 E746V | 15.3 |

From the above results, it was revealed that a combination of a primer and a BNA oligonucleotide as used in this experiment attained a mutation concentration effect even in detection of other deletion mutations present in the same genome region as that of L747T751>S.

### Example 3

The effect of suppressing amplification of a wild-type nucleic acid was examined using a BNA oligonucleotide with the same base sequence as that of the BNA oligo (E19B4) used in Example 1 and with binding strength improved by increasing the number of nucleosides having a BNA structure. In addition, the relationship between the amplification suppression effect and the number of bases between the primer and BNA oligonucleotide was examined. Among the combinations of primers used in Example 1, three combinations: E19F1-delR3. E19F1-E19R1 and E19F1-E19R2 were employed. As template DNAs, EG_19 wt as a wild-type plasmid and EG_de1746_750(2235) as a mutant-type plasmid were used. Table 6 shows the base sequences of a newly used BNAoligo (E19B9).

TABLE 6

| | | SEQ |
|---|---|---|
| | | ID |
| | Base sequence | NO. |
| BNA oligo | 5′-GT(H)T(H)GCT(H)T(H)5(H)T(H)5(H) | (22) |
| (E19B9) | TT(H)AA(E)TT(H)5(H)Cp | |

TABLE 7

| | | | Number of bases between |
|---|---|---|---|
| Forward primer | Reverse primer | ΔΔCt | reverse primer and BNA oligo |
| E19F1 | delR3 | 22.8 | 5 |
| E19F1 | E19R2 | 10.6 | 33 |
| E19F1 | E19R1 | 10.3 | 72 |

Table 7 shows the measured ΔΔCt value, and the number of bases between the 5′ terminal of the BNA oligo (E19B9) and the 3′ terminal of the reverse primer on the template DNA (“the number of bases between the reverse primer and the BNA oligo” in the table). These results showed that since the BNA oligo (E19B9) with higher binding strength was used, the ΔΔCt value was higher as compared with the result in Example 1 in all the cases, and a high effect of suppressing amplification of a wild-type nucleic acid was obtained as a whole. Further, as with Example 1, an evidently higher effect of suppressing amplification of a wild-type nucleic acid was obtained when the distance between the reverse primer and the BNA oligonucleotide was 5 bases than when the distance was 33 bases or 72 bases. These results showed that for the mutation concentration effect by the BNA oligonucleotide, the distance between the BNA oligonucleotide and a primer that is hybridized with the same single-stranded template DNA was extremely important irrespective of the kind of BNA oligonucleotide.

### Example 4

Using the primers and BNA oligonucleotide shown in Table 8, the mutation concentration effect (ΔΔCt) by PCR-clamping in detection of a genetic mutation group in a deletion region containing de1752_759(2253) as a deletion mutation in exon 19 of hEGFR was examined. SEQ ID NO: 25 is the number of a base sequence before the BNA oligo (S752-BNA1) is subjected to various kinds of modifications.

As template DNAs, EG_19 wt as a wild-type plasmid and EG_de1752_759(2253) as a mutant-type plasmid were used. The forward primer is hybridized with a single-stranded template DNA with which a BNA oligonucleotide is hybridized, and the reverse primer is hybridized with a single-stranded template DNA complementary to the single-stranded template DNA with which the BNA oligonucleotide is hybridized.

TABLE 8

| | Base sequence | SEQ ID NO. |
|---|---|---|
| Forward primer | 5'-TCTCTGTCATAGGGACTCTGGA | 17 |
| (E19F1) | | |
| Forward primer | 5'-ATCAAGGAATTAAGAGAAGCAAC | 23 |
| (S752F1) | | |
| Reverse primer | 5'-GAGAAAAGGTGGGCCTGAGG | 19 |
| (E19R1) | | |
| Reverse primer | 5'-GCAGAAACTCACATCGAG | 74 |
| (S752R2) | | |
| BNA oligo | 5'-T(H)5(H)5(H)GA(E)A(E)A(E)G5 | (25) |
| (S752-BNA1) | (H)5(H)AA(E)p | |

Table 9 shows combinations of primers, and ΔΔCt values when the BNA oligo (S752-BNA1) is used. In Table 9, the “wild-type ΔCt” means ΔCt when the wild-type plasmid EG_19 wt is used, and the “mutant-type ΔCt” means ΔCt when the mutant-type plasmid EG_de1752_759(2253) is used.

TABLE 9

| | | ΔCt for | ΔCt for | |
|---|---|---|---|---|
| Forward primer | Reverse primer | wild-type | mutant-type | ΔΔCt |
| E19F1 | E19R1 | 0 | 0.1 | −0.1 |
| S752F1 | S752R2 | 12.7 | 0.4 | 12.3 |
| E19F1 | S752R2 | 0.3 | 0.1 | 0.2 |
| S752F1 | E19R1 | 14.3 | 1.1 | 13.2 |

From these results, it was revealed that the mutation concentration effect by the BNA oligo (S752-BNA1) was dramatically influenced by a combination of primers. It was found that only when the forward primer was S752F1, a large ΔΔCt value of 10 or more was obtained, a mutation concentration effect was exhibited, and the mutation concentration effect was not influenced by the reverse primer. On the template DNA, the 5′ terminal of the BNA oligo (S752-BNA1) is separated from the 3′ terminal of the forward primer S752F1 by 3 bases, and separated from the 3′ terminal of the forward primer E19F1 by 61 bases. In addition, on the template DNA, the base complementary to the 3′ terminal of the BNA oligo (S752-BNA1) is separated from the 3′ terminal of the reverse primer S752R2 by 10 bases, and separated from the 3′ terminal of the reverse primer E19R1 by 57 bases. That is, when the distance between the 5′ terminal of the BNA oligo (S752-BNA1) and the 3′ terminal of the primer that is hybridized with a single-stranded template DNA identical to the single-stranded template DNA with which the BNA oligo (S752-BNA1) is hybridized was 61 bases, the mutation concentration effect by the BNA oligo (S752-BNA1) was not obtained at all, and when the distance was 3 bases, a remarkable mutation concentration effect was obtained. On the other hand, it was revealed that the number of bases between the BNA oligo (S752-BNA1) and the primer that is hybridized with a single-stranded template DNA complementary to the single-stranded template DNA with which the BNA oligo (S752-BNA1) is hybridized did not influence the mutation concentration effect by the BNA oligo (S752-BNA1) at all. That is, these results showed that for the mutation concentration effect by the BNA oligonucleotide, the distance between the BNA oligonucleotide and a primer that is hybridized with the same single-stranded template DNA was extremely important.

In addition, using a primer set (forward primer S752F1 and reverse primer E19R1) and a BNA oligo (S752-BNA1), the mutation concentration effect was examined for another deletion mutation de1752_759(2254) present in the same genome region as that of de1752_759(2253) was examined. The result showed that the ΔΔCt value was 10.4, and thus the same mutation concentration effect as in the case of the deletion mutation de1752-759(2253) was obtained. These results showed that for improving the mutation concentration effect by the BNA oligo (S752-BNA1), the position of a primer that is hybridized with a single-stranded template DNA identical to that of the BNA oligo (S752-BNA1) is preferably close to the BNA oligo (S752-BNA1).

### Example 5

Using the primers and BNA oligonucleotide shown in Table 10, the mutation concentration effect (ΔΔCt) by PCR-clamping in detection of T790M which is a single-base mutation in exon 20 of hEGFR was examined. SEQ ID NO: 28 is the number of a base sequence before the BNA oligo (T790-BNA2) is subjected to various kinds of modifications.

As template DNAs, EG_20 wt as a wild-type plasmid and EG_T790M as a mutant-type plasmid were used. The forward primer is hybridized with a single-stranded template DNA with which a BNA oligonucleotide is hybridized, and the reverse primer is hybridized with a single-stranded template DNA complementary to the single-stranded template DNA with which the BNA oligonucleotide is hybridized.

TABLE 10

| | Base sequence | SEQ ID NO. |
|---|---|---|
| Forward primer | 5'-ATCTGCCTCACCTCCACCGT | 26 |
| (E20F5) | | |
| Reverse primer | 5'-CCCGTATCTCCCTTCCCTGA | 27 |
| (E20R1) | | |
| BNA oligo | 5'-T5(H)A(E)5(H)(E)5(H)A(E)G5 | |
| (T790-BNA2) | (H)T(H)CAp | (28) |

The result showed that the ΔΔCt value was 13.4, and thus a mutation concentration effect was obtained.

In this example, the 5′ terminal of the BNA oligo (T790-BNA2) is separated from the 3′ terminal of the forward primer E20F5 by 8 bases on the template DNA.

### Example 6

Using the primers and BNA oligonucleotide shown in Table 11, the mutation enrichment effect (ΔΔCt) by PCR-clamping in detection of G719A, G719S and G719C which are single-base mutations in exon 18 of hEGFR was examined. SEQ ID NO: 31 is the number of a base sequence before the BNA oligo (G719-BNA1) is subjected to various kinds of modifications.

As template DNAs, EG_G719 wt as a wild-type plasmid, EG_G719C as a G719C mutant-type plasmid, EG_G719S as a G719S mutant-type plasmid, and EG_G719A as a G719A mutant-type plasmid were used. The reverse primer is hybridized with a single-stranded template DNA with which a BNA oligonucleotide is hybridized, and the forward primer is hybridized with a single-stranded template DNA complementary to the single-stranded template DNA with which the BNA oligonucleotide is hybridized.

TABLE 11

| | Base sequence | SEQ ID NO. |
|---|---|---|
| Forward primer | 5'-CCAACCAAGCTCTCTTGAGG | 29 |
| (E18F1) | | |
| Reverse primer | 5'-GTGCCAGGGACCTTACCTTA | 30 |
| (E18R1) | | |
| BNA oligo | 5'-5(H)5(H)G(E)GA(E)G(E)5(H)5(H)5 | (31) |
| (G719-BNA1) | (H)A(E)Gp | |

TABLE 12

| Plasmid as template | ΔΔCt | Mutation enrichment ratio |
|---|---|---|
| G719C | 14.2 | 18,800 |
| G719S | 15.5 | 46,300 |
| G719A | 16.8 | 114,000 |

Table 12 shows the obtained ΔΔCt values and the mutation enrichment ratios (ΔΔCt power of 2 [2^{ΔΔCt}]). These results showed that a combination of a primer and a BNA oligonucleotide as used in this experiment attained a large ΔΔCt value for any mutation. In addition, it was found that the mutation concentration ratio was 18,000 or more, and thus a large PCR-clamping effect was obtained. In this example, the 5′ terminal of the BNA oligo (G719-BNA1) is separated from the 3′ terminal of the reverse primer E18R1 by 18 bases on the template DNA.

### Example 71

Using the primers and BNA oligonucleotide shown in Table 13, the mutation enrichment effect (ΔΔCt) by PCR-clamping in detection of L858R and L861Q which are single-base mutations in exon 21 of hEGFR was examined. SEQ ID NO: 36 is the number of a base sequence before the BNA oligo (E21B6) is subjected to various kinds of modifications.

As template DNAs, EG-21 wt as a wild-type plasmid, EG_L858R as a L858R mutant-type plasmid, and EG_L861Q as a L861Q mutant-type plasmid were used. The reverse primer is hybridized with a single-stranded template DNA with which a BNA oligonucleotide is hybridized, and the forward primer is hybridized with a single-stranded template DNA complementary to the single-stranded template DNA with which the BNA oligonucleotide is hybridized.

TABLE 13

| | Base sequence | SEQ ID NO. |
|---|---|---|
| Forward primer | 5'-AGCCAGGAACGTACTGGTGA | 32 |
| (E21F1) | | |
| Forward primer | 5'-TTGCAGCATGTCAAGATCACAGATTTTG | 33 |
| (858F5) | | |
| Reverse primer | 5'-AAAGCCACCTCCTTACTTTGC | 34 |
| (E21R1) | | |
| Reverse primer | 5'-AACTTTCTCTTCGGCACCCA | 35 |
| (858R2) | | |
| BNA oligo | 5'-CAG5(H)AGT(H)TTGGCCA(E)GCCCAp | (36) |
| (E21B6) | | |

Table 14 shows combinations of primers, and ΔΔCt values when the BNA oligo (E21B6) is used. In Table 14, “ΔΔCt (L858R)” denotes a ΔΔCt value when a L858R mutation is detected, and “ΔΔCt (L861Q)” denotes a ΔΔCt value when a L861Q mutation is detected.

TABLE 14

| Forward primer | Reverse primer | ΔΔCt (L858R) | ΔΔCt (L861Q) |
|---|---|---|---|
| E21F1 | E21R1 | 0.1 | −0.1 |
| 858F5 | 858R2 | 9.2 | 13.5 |
| E21F1 | 858R2 | 9.2 | 13.5 |

These results showed that only when the reverse primer was 858R2, a large ΔΔCt value of about 10 was obtained, a mutation enrichment effect was exhibited, and the mutation enrichment effect was not influenced by the forward primer for any mutation. In this example, on the template DNA, the 5′ terminal of the BNA oligo (E21B6) overlaps the 3′ terminal of the reverse primer 858R2 by 2 bases, and is separated the 3′ terminal of the reverse primer 858R2 by 34 bases. That is, when the distance between the 5′ terminal of the BNA oligo (E21B6) and the 3′ terminal of the primer that is hybridized with a single-stranded template DNA identical to the single-stranded template DNA with which the BNA oligo (E21B6) is hybridized was 34 bases, the mutation enrichment effect by the BNA oligo (E21B6) was not obtained at all. On the other hand, it was found that even when both the terminals overlapped each other by 2 bases, a sufficient mutation concentration effect was obtained.

In Examples 1 to 7, a BNA oligonucleotide was used as a wild-type oligonucleotide. However, each of LNA and BNA is a non-natural nucleic acid in which 2′-oxygen and 4′-carbon in the ribose ring of the ribonucleoside are linked to each other, and LNA and BNA have the same PCR-clamping action mechanism. Therefore, the positional relationship between the BNA oligonucleotide and the primer can be directly applied to the positional relationship between the LNA oligonucleotide and the primer.

### INDUSTRIAL APPLICABILITY

According to the present invention, an extremely small amount of a mutant-type gene contained in a highly excessive amount of a wild-type gene can be detected with high sensitivity. Therefore, the method for detecting a genetic mutation according to the present invention is useful particularly in clinical examinations.

1. A method for detecting a genetic mutation, the method comprising:
carrying out PCR using a template DNA comprising a target genetic mutation site, a forward primer and a reverse primer each configured to amplify a region containing the target genetic mutation site, and a wild-type oligonucleotide comprising a base sequence complementary to wild type template DNA; and
detecting an amplified DNA comprising a mutant-type genetic mutation site on the basis of the result of PCR amplification,
wherein the wild-type oligonucleotide comprises LNA or BNA, and
wherein on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

2. The method according to claim 1, wherein the wild-type oligonucleotide comprises BNA.

3. The method according to claim 1, wherein
in the PCR amplification, a nucleic acid chain-extending reaction may be carried out using a DNA polymerase at a temperature at which the wild-type oligonucleotide is hybridized with a wild type template DNA comprising a wild-type genetic mutation site, and the wild-type oligonucleotide is substantially not hybridized with a mutant type template DNA comprising a mutant-type genetic mutation site.

4. The method according to claim 1, wherein the PCR is real-time PCR.

5. The method according to claim 1, wherein the region with which the wild-type oligonucleotide is a hybridized is located between a region with which the forward primer is a hybridized and a region with which the reverse primer is a hybridized

6. The method according to claim 1, wherein on a DNA strand of the template, with which the wild-type oligonucleotide is hybridized, a region with which the wild-type oligonucleotide is a hybridized and a region with which the forward primer or the reverse primer is hybridized are separated from each other by 1 to 18 bases.

7. The method according to claim 1, wherein on a DNA strand of the template, with which the wild-type oligonucleotide is hybridized, a region with which the wild-type oligonucleotide is a hybridized and a region with which the reverse primer is hybridized partially overlap each other, or the regions are separated from each other by 1 to 18 bases.

8. A method for detecting a genetic mutation, the method comprising:
providing a sample comprising mutant type template DNA and wild type template DNA, wherein the mutant type template DNA comprises a mutant-type genetic mutation site, and the wild type template DNA comprises a wild-type genetic mutation site,
selectively amplifying the mutant type template DNA in the sample using a forward primer and a reverse primer each configured to amplify a region containing the mutant type genetic mutation site, and a wild-type oligonucleotide comprising a base sequence complementary to wild type template DNA; and
detecting an amplified DNA comprising a mutant-type genetic mutation site on the basis of the result of PCR amplification,
wherein the wild-type oligonucleotide comprises LNA or BNA, and
wherein on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

9. The method according to claim 8, wherein the wild-type oligonucleotide comprises BNA.

10. The method according to claim 8, wherein
in the amplification, a nucleic acid chain-extention reaction may be carried out using a DNA polymerase at a temperature at which the wild-type oligonucleotide is hybridized with the wild type template DNA comprising the wild-type genetic mutation site, and the wild-type oligonucleotide is substantially not hybridized with the mutant type template DNA comprising the mutant-type genetic mutation site.

11. The method according to claim 8, wherein the amplification is real-time PCR.

12. The method according to claim 8, wherein the region with which the wild-type oligonucleotide is a hybridized is located between a region with which the forward primer is a hybridized and a region with which the reverse primer is a hybridized

13. The method according to claim 8, wherein on a DNA strand of the template, with which the wild-type oligonucleotide is hybridized, a region with which the wild-type oligonucleotide is a hybridized and a region with which the forward primer or the reverse primer is hybridized are separated from each other by 1 to 18 bases.

14. The method according to claim 8, wherein on a DNA strand of the template, with which the wild-type oligonucleotide is hybridized, a region with which the wild-type oligonucleotide is a hybridized and a region with which the reverse primer is hybridized partially overlap each other, or the regions are separated from each other by 1 to 18 bases.

15. A method for amplifying DNA, the method comprising:
providing a sample comprising mutant template DNA and wild type template DNA, wherein the mutant template DNA comprises a mutant-type sequence comprising a mutation, and the wild type template DNA comprises a wild-type sequence, and
selectively amplifying the mutant template DNA in the sample using, a forward primer and a reverse primer each configured to amplify a region containing the mutation, and a wild-type oligonucleotide complementary to wild type sequence;
wherein the wild-type oligonucleotide comprises LNA or BNA, and
wherein on a DNA strand of the template, with which the wild-type oligonucleotide is hybridized, a region with which the wild-type oligonucleotide is a hybridized and a region with which the forward primer or the reverse primer is hybridized partially overlap each other, or the regions are separated from each other by 1 to 18 bases.

16. The method according to claim 15, wherein the wild-type oligonucleotide comprises BNA.

17. The method according to claim 15, wherein
in the amplification, a nucleic acid chain-extending reaction may be carried out using a DNA polymerase at a temperature at which the wild-type oligonucleotide is hybridized with the wild type template DNA comprising the wild-type genetic mutation site, and the wild-type oligonucleotide is substantially not hybridized with the mutant type template DNA comprising the mutant-type genetic mutation site.

18. The method according to claim 15, wherein the amplification is real-time PCR.

19. The method according to claim 15, wherein the region with which the wild-type oligonucleotide is a hybridized is located between a region with which the forward primer is a hybridized and a region with which the reverse primer is a hybridized

20. The method according to claim 15, wherein on a DNA strand of the template, with which the wild-type oligonucleotide is hybridized, a region with which the wild-type oligonucleotide is a hybridized and a region with which the forward primer or the reverse primer is hybridized are separated from each other by 1 to 18 bases.


SEQUENCE LISTING (36 sequences)

SEQ ID NO: 1 - DNA, 300 bp, Homo sapiens
ccttgtctctgtgttcttgtcccccccagcttgtggagcctcttacacccagtggagaagctcccaaccaagctctcttgaggatcttgaaggaaactgaattcaaaaagatcaaagtgctgggctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttccc

SEQ ID NO: 2 - DNA, 300 bp, Homo sapiens
ccttgtctctgtgttcttgtcccccccagcttgtggagcctcttacacccagtggagaagctcccaaccaagctctcttgaggatcttgaaggaaactgaattcaaaaagatcaaagtgctgtgctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttccc

SEQ ID NO: 3 - DNA, 300 bp, Homo sapiens
ccttgtctctgtgttcttgtcccccccagcttgtggagcctcttacacccagtggagaagctcccaaccaagctctcttgaggatcttgaaggaaactgaattcaaaaagatcaaagtgctgagctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttccc

SEQ ID NO: 4 - DNA, 300 bp, Homo sapiens
ccttgtctctgtgttcttgtcccccccagcttgtggagcctcttacacccagtggagaagctcccaaccaagctctcttgaggatcttgaaggaaactgaattcaaaaagatcaaagtgctggcctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttccc

SEQ ID NO: 5 - DNA, 300 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 6 - DNA, 285 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaagacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 7 - DNA, 285 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaaaacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 8 - DNA, 291 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaaggaaccaacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 9 - DNA, 282 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaaggttccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 10 - DNA, 282 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaaggaatcgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 11 - DNA, 276 bp, Homo sapiens
acatccacccagatcactgggcagcatgtggcaccatctcacaattgccagttaacgtcttccttctctctctgtcatagggactctggatcccagaaggtgagaaagttaaaattcccgtcgctatcaaggaattaagagaagcaaccctcgatgtgagtttctgctttgctgtgtgggggtccatggctctgaacctcaggcccaccttttctcatgtctggcagctgctctgctctagaccctgctcatctccacatcctaaatgttcacttt

SEQ ID NO: 12 - DNA, 300 bp, Homo sapiens
ccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctc

SEQ ID NO: 13 - DNA, 300 bp, Homo sapiens
ccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctc

SEQ ID NO: 14 - DNA, 300 bp, Homo sapiens
tgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccaga

SEQ ID NO: 15 - DNA, 300 bp, Homo sapiens
tgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccaga

SEQ ID NO: 16 - DNA, 300 bp, Homo sapiens
tgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaacagctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccaga

SEQ ID NO: 17 - DNA, 22 bp, Artificial Sequence
  Synthetic oligonucleotide primer (E19F1)
tctctgtcatagggactctgga

SEQ ID NO: 18 - DNA, 18 bp, Artificial Sequence
  Synthetic oligonucleotide primer (delF7)
agttaaaattcccgtcgc

SEQ ID NO: 19 - DNA, 20 bp, Artificial Sequence
  Synthetic oligonucleotide primer (E19R1)
gagaaaaggtgggcctgagg

SEQ ID NO: 20 - DNA, 20 bp, Artificial Sequence
  Synthetic oligonucleotide primer (delR3)
gatttccttgttggctttcg

SEQ ID NO: 21 - DNA, 18 bp, Artificial Sequence
  Synthetic oligonucleotide primer (E19R2)
agagcaaaggagaaactc

SEQ ID NO: 22 - DNA, 18 bp, Artificial Sequence
  Synthetic oligonucleotide, BNA oligo (E19B4) or BNA oligo (E19B9)
gttgcttctcttaattcc

SEQ ID NO: 23 - DNA, 23 bp, Artificial Sequence
  Synthetic oligonucleotide primer (S752F1)
atcaaggaattaagagaagcaac

SEQ ID NO: 24 - DNA, 18 bp, Artificial Sequence
  Synthetic oligonucleotide primer (S752R2)
gcagaaactcacatcgag

SEQ ID NO: 25 - DNA, 12 bp, Artificial Sequence
  Synthetic oligonucleotide, BNA oligo (S752-BNA1)
tccgaaagccaa

SEQ ID NO: 26 - DNA, 20 bp, Artificial Sequence
  Synthetic oligonucleotide primer (E20F5)
atctgcctcacctccaccgt

SEQ ID NO: 27 - DNA, 20 bp, Artificial Sequence
  Synthetic oligonucleotide primer (E20R1)
cccgtatctcccttccctga

SEQ ID NO: 28 - DNA, 12 bp, Artificial Sequence
  Synthetic oligonucleotide, BNA oligo (T790-BNA2)
tcacgcagctca

SEQ ID NO: 29 - DNA, 20 bp, Artificial Sequence
  Synthetic oligonucleotide primer (E18F1)
ccaaccaagctctcttgagg

SEQ ID NO: 30 - DNA, 20 bp, Artificial Sequence
  Synthetic oligonucleotide primer (E18R1)
gtgccagggaccttacctta

SEQ ID NO: 31 - DNA, 11 bp, Artificial Sequence
  Synthetic oligonucleotide, BNA oligo (G719-BNA1)
ccggagcccag

SEQ ID NO: 32 - DNA, 20 bp, Artificial Sequence
  Synthetic oligonucleotide primer (E21F1)
agccaggaacgtactggtga

SEQ ID NO: 33 - DNA, 28 bp, Artificial Sequence
  Synthetic oligonucleotide primer (858F5)
ttgcagcatgtcaagatcacagattttg

SEQ ID NO: 34 - DNA, 21 bp, Artificial Sequence
  Synthetic oligonucleotide primer (E21R1)
aaagccacctccttactttgc

SEQ ID NO: 35 - DNA, 20 bp, Artificial Sequence
  Synthetic oligonucleotide primer (858R2)
aactttctcttccgcaccca

SEQ ID NO: 36 - DNA, 19 bp, Artificial Sequence
  Synthetic oligonucleotide, BNA oligo (E21B6)
cagcagtttggccagccca